(19) United States
(12) Patent Application Publication
(10) Pub. No.: US 2005/0037499 A1
(43) Pub. Date: Feb. 17, 2005

(54) Apparatus and method for determining temperatures at which properties of materials change

(75) Inventors: C. Eric Ramberg, San Jose, CA (US); Youqi Wang, Atherton, CA (US); Shenheng Guan, Palo Alto, CA (US); David Lee, La Mirada, CA (US); Sum Nguyen, Fremont, CA (US)

Correspondence Address: SYMYX TECHNOLOGIES INC;LEGAL DEPARTMENT, 3100 CENTRAL EXPRESS, SANTA CLARA, CA, 95051, US

(73) Assignee: Symyx Technologies, Inc.

(21) Appl. No.: 10/639,003

(22) Filed: Aug. 11, 2003

(51) Int. Cl. G01N 33/00; G01N 33/48

(52) U.S. Cl. 436/3; 422/68.1

(57) ABSTRACT

An analytical measurement system includes a sample holder, a temperature changing device for changing the temperature of a sample on the sample holder, and a measurement device for detecting a change in the sample such as a phase change. The sample holder can be a micro sample holder including a support member having an aperture, a membrane spanning the aperture, and a backing layer attached to a backside of the membrane. The temperature changing device can be a laser directed at the backside of the sample holder or an electrical resistive heater on the sample holder. The measurement device senses changes in the sample such as the film stress, viscosity, or surface reflectivity, by measuring changes in scattered light, reflected light, emitted light, or electrical resistance.

### CROSS REFERENCE TO RELATED APPLICATIONS

This application is related to U.S. patent application titled “APPARATUS AND METHOD FOR DETECTING THERMOELECTRIC PROPERTIES OF MATERIALS” attorney docket number 2000-107, filed on the same date as this application.

### FIELD OF THE INVENTION

The present invention relates to the field of materials analysis and more particularly to methods and apparatus for rapidly screening materials for phase transition temperatures.

### BACKGROUND OF THE INVENTION

Combinatorial materials science seeks to rapidly screen large numbers of materials for important characteristics in order to discover commercially valuable new materials (see, for example, U.S. Pat. No. 6,004,617 issued to Schultz, et al. which is incorporated herein by reference). One characteristic that is used to evaluate prospective materials is a phase transition temperature. A phase transition temperature is the temperature at which a material undergoes a particular phase change. Examples include the melting point, the temperature at which a solid material transforms to a liquid phase; the solidification temperature, the temperature at which a liquid transforms to a solid phase; the crystallization temperature, the temperature at which a material transforms from an amorphous phase to a crystalline phase; and solid-state phase transitions between different crystalline phases of the same material. Many other phase transitions are known.

Techniques to determine phase transition temperatures include differential scanning calorimetry (DSC) and differential thermal analysis (DTA). A sample in a DSC analysis is heated or cooled at a specific rate, for example 10° C. per minute, and the amount of energy required to maintain the steady temperature increase or decrease is plotted as a function of temperature. Since phase transitions tend to be either exothermic or endothermic, the plot of energy against temperature will show either a drop or a rise at a phase transition temperature, or more commonly, in a narrow temperature range around the phase transition temperature. In a DTA analysis a temperature sensing device such as a thermocouple is placed in an inert reference material and another is placed in a sample of the material under study. The temperature difference between the two thermocouples is monitored while both materials are identically heated. A deflection of the temperature difference between the two thermocouples indicates that the sample is undergoing a phase transition or a reaction. This occurs because the phase transition causes the temperature of the sample to change either more or less quickly than the temperature of the reference.

In order to screen a large number of materials efficiently, typically only a small quantity of each material is prepared. These materials can be arranged in an array on a common substrate for parallel or rapid serial processing and evaluation. DSC and DTA require significant amounts of sample material and are therefore not well suited for combinatorial screening methods. However, techniques for determining phase transition temperatures have been adapted to the sample constraints of the combinatorial approach.

For example, U.S. Pat. No. 6,536,944 issued to Archibald, et al. discloses a library of materials (i.e., an array of compositionally varying samples) on a common substrate simultaneously heated by a common heater beneath the substrate. A detector, such as an infra-red detector, situated above the substrate monitors changes in radiation from the samples, for example changes in reflectivity or emissivity, as a function of temperature. The radiation from the substrate is measured along with the radiation from the samples. A calibration curve allows the temperature of the substrate to be determined from the measured radiation.

A similar scheme, illustrated in U.S. Pat. No. 6,541,271 issued to McFarland, et al. discloses a library of samples on an infra-red transparent substrate. The samples are heated from above by an infra-red source either individually or simultaneously. An infra-red detector views the radiation from the samples through the transparent substrate also either individually or simultaneously.

U.S. Pat. Nos. 5,345,213, 5,356,756, and 5,464,966 respectively issued to Semancik, et al., Cavicchi, et al., and Gaitan, et al. disclose a micro-hotplate sensor that can be used for determining phase transition temperatures. Each micro-hotplate consists of a membrane suspended above an etch pit in a surrounding substrate, a heating element on top of the membrane, and a conductive heat distribution plate over and electrically isolated from the heating element. The heat distribution plate includes electrical leads for temperature sensing and control. The sample is placed above the heat distribution plate and in contact with additional electrical contact pads that allow the electrical resistance of the sample to be measured. While the membrane serves to at least partially thermally isolate the sample from the surrounding substrate, the electrical leads for temperature sensing and control, and the leads to the contact pads, all provide thermally conductive paths that allow heat to bleed away from the sample.

U.S. Pat. Nos. 6,438,497, 6,477,479, and 6,535,824 all issued to Mansky, et al., and U.S. Patent Publication No. 2002-0032531 also to Mansky et al., disclose a library of materials on a common substrate. In these references each sample is situated within a well in the substrate and on a membrane that forms the bottom of the well. Circuitry for heating and temperature measurements are printed on the opposite side of each membrane.

Some phase transitions of interest, however, are not easily observed by the forgoing methods. For example, an amorphous material heated beyond the glass transition temperature will transform from a generally brittle phase to a more elastic phase. This transition typically does not produce a significant change in electrical resistance nor a significant change in enthalpy that would be detected by monitoring temperature such as with an infra-red detector. Therefore, a need exists for methods and apparatus to rapidly screen samples of a combinatorial library for phase transition temperatures of a variety of phase transitions.

### SUMMARY OF THE INVENTION

The present invention provides a resolution to these needs. The invention discloses an analytical measurement system that includes a micro sample holder for supporting a sample, a temperature changing device configured to change a temperature of the sample over a measurement period of time, and a measurement device configured to measure a change in a property of the sample over the measurement period. The micro sample holder includes a support member having an aperture disposed therein, a membrane spanning the aperture and including a sample region that is centrally located in some embodiments, and a thermally conductive backing layer. The membrane includes a first surface for supporting the sample on the sample region, and a second surface opposite the first surface. The thermally conductive backing layer is in thermal communication with the second surface and is contiguous with the sample region of the membrane, and in some embodiments includes a coating for absorbing light. In some embodiments a protective film is disposed over the membrane to prevent chemical reactions between the sample and the membrane.

In some embodiments at least two electrical contacts are disposed on the first surface for electrical communication with the sample, and in some of these embodiments the temperature changing device is configured to pass a current, which can be modulated, between the at least two electrical contacts to heat the sample. Also in some embodiments the measurement device is in electrical communication with the at least two electrical contacts and the change in the property of the sample that is measured by the measurement device includes a change in electrical resistance of the sample.

In some embodiments the membrane forms a bridge between opposing sides of the aperture. Also, the membrane can further include a suspension having at least two segments disposed between the sample region and the support member. The at least two segments can include a plurality of perforations in some embodiments.

The measurement device in some embodiments measures the change in the property of the sample by recording the property as a function of time, or as a function of temperature, as the temperature of the sample is changed. The temperature changing device can include a resistive heater, or a laser configured to direct a laser beam at the backing layer. In the latter embodiments, the laser can be configured to modulate the laser beam.

The measurement device can be configured to record illumination scattered from the sample, reflected from the sample, or emitted by the sample or by the backing layer. The measurement device can also include a mechanical oscillator affixed to the support member to harmonically induce a vibration in the sample. In some embodiments the mechanical oscillator is tunable to vary a frequency of the harmonically induced vibration.

Another analytical measurement system of the invention includes a sample holder including a first surface having at least two electrical contacts disposed thereon for electrical communication with a sample disposed on the first surface and a second surface opposite the first surface, a laser configured to heat the sample through a temperature range, and a measurement device configured to measure the electrical resistance of the sample while the sample is heated through the temperature range. In some of these embodiments the laser is configured to heat the sample by directing a laser beam at the second surface of the sample holder. Also in some embodiments the measurement device includes an infra-red detector configured to receive radiation emitted from the sample holder to measure a temperature thereof.

Still another analytical measurement system of the invention includes a sample holder including a first surface for supporting a sample disposed thereon and a second surface opposite the first surface, a first laser configured to heat the sample through a temperature range, and a measurement device. In these embodiments the measurement device includes a second laser configured to irradiate the sample, and a detector configured to receive radiation emanating from the irradiated sample to detect a change in the sample as the sample is heated through the temperature range. In some of these embodiments the first laser is configured to heat the sample by directing a first laser beam at the second surface of the sample holder. The radiation emanating from the irradiated sample can include reflected radiation. The detector can be configured to detect the change in the sample by determining a change in a stress of the sample, or by determining a change in a viscosity of the sample. In some embodiments the second laser is configured to irradiate the sample with an array of laser beams, and in some of these embodiments the detector is a line camera detector.

Yet another analytical measurement system of the invention includes a sample holder including a first surface for supporting a sample disposed thereon and a second surface opposite the first surface, a laser configured to heat the sample through a temperature range, and a measurement device. In these embodiments the measurement device includes a light source configured to illuminate the sample, and a detector configured to receive radiation emanating from the illuminated sample to detect a change in the sample as the sample is heated through the temperature range. In some of these embodiments the detector is a video camera. The light source can produce, in some embodiments, an illumination with a wavelength shorter than that of infrared radiation, and in some embodiments the light source is a blue LED.

A method of the invention is directed to determining a change in a property of a material. The method includes providing a micro sample holder including a support member having an aperture disposed therein, a membrane spanning the aperture and having a sample region and also having a first surface and a second surface opposite the first surface, and a thermally conductive backing layer in thermal communication with the second surface and contiguous with the sample region of the membrane. The method further includes synthesizing a sample of the material on the first surface of the sample holder within the sample region, affecting the environment of the sample while measuring a property of the sample to generate a record thereof, and analyzing the record for a change in the property. In these embodiments affecting the environment of the sample can include changing the temperature of the thermally conductive backing layer, changing the pressure of an atmosphere surrounding the sample, or changing the composition of an atmosphere surrounding the sample.

In some embodiments measuring the property of the sample includes imaging an appearance of the sample, and in some of these embodiments analyzing the record for the change in the property includes correlating a change in the appearance to a phase change. In some embodiments measuring the property of the sample includes monitoring a deformation of the sample, and in some of these embodiments analyzing the record for the change in the property includes correlating a change in deformation of the sample to a phase change, or correlating a change in deformation of the sample to a change in a composition of the sample. In some embodiments measuring the property of the sample includes measuring a vibration of the sample, and in some of these embodiments analyzing the record for the change in the property includes correlating a change in the vibration of the sample to a phase change, correlating a change in the vibration of the sample to a change in the viscosity of the sample, or correlating a change in the vibration of the sample to a change in the mass of the sample. In some embodiments measuring the property of the sample includes measuring a reflectivity of the sample, and in some of these embodiments analyzing the record for the change in the property includes correlating a change in the reflectivity of the sample to a phase change. In some embodiments measuring the property of the sample includes measuring an electrical resistance of the sample, in some of these embodiments analyzing the record for the change in the property includes correlating a change in the electrical resistance of the sample to a phase change.

Another method of the invention is directed to determining a change in a property of a material. This method includes providing a sample holder including a sample region having opposing first and second surfaces, synthesizing a sample of the material on the first surface of the sample region, heating the sample region of the sample holder by directing a laser beam towards the second surface while measuring an electrical resistivity of the sample to generate a record thereof, and analyzing the record for a change in the electrical resistivity.

Further objects and aspects of this invention will be evident to those of skill in the art upon review of this specification.

### BRIEF DESCRIPTION OF THE DRAWINGS

FIG. 1 is a cross-sectional view of a micro sample holder according to an embodiment of the invention.

FIG. 2 is a bottom plan view of the micro sample holder shown in FIG. 1.

FIG. 3 is a cross-sectional view of a micro sample holder according to another embodiment of the invention.

FIG. 4 is a cross-sectional view of a micro sample holder according to another embodiment of the invention.

FIG. 5 is a cross-sectional view of a micro sample holder according to another embodiment of the invention.

FIG. 6 is a top plan view of the micro sample holder shown in FIG. 5.

FIG. 7 is a perspective view of a micro sample holder according to another embodiment of the invention.

FIG. 8 is a perspective view of a micro sample holder according to another embodiment of the invention.

FIG. 9 is a perspective view of a micro sample holder according to another embodiment of the invention.

FIG. 10 is a perspective view of a micro sample holder according to another embodiment of the invention.

FIG. 11 is a top plan view of a micro sample holder according to another embodiment of the invention.

FIGS. 12 and 13 are cross-sectional views illustrating an exemplary method of the invention for fabricating a micro sample holder.

FIGS. 14-18 illustrate another exemplary method of the invention for fabricating a micro sample holder.

FIG. 19 is a top plan view of a micro sample holder according to another embodiment of the invention.

FIG. 20 is a schematic representation of a analytical measurement system according to an embodiment of the invention.

### DETAILED DESCRIPTION OF THE INVENTION

The present invention is directed to sample holders of an appropriate scale for supporting individual samples on a substrate. The invention is also directed to arrays of such sample holders formed on, or integral with, a common substrate. The invention is further directed to methods for making the sample holders, analytical measurement systems that can incorporate the sample holders, and methods for using the analytical measurement systems to determine phase transition temperatures and to monitor other changes to the sample as a function of temperature.

More particularly, a sample holder of the invention can include a suspended membrane having a sample region portion on which the sample rests. The sample holder can also include a thermally conductive backing layer on a side of the sample region opposite the side that supports the sample. The backing layer serves to distribute heat evenly to the sample so that the sample is essentially at a uniform temperature at any given moment, even during rapid heating and cooling. The backing layer also serves to reinforce the sample region of the membrane against deforming or breaking in response to stresses created by the sample. It will be understood, however, that the backing layer does not completely eliminate deformation in response to stresses, and in some instances the methods for using the analytical measurement systems rely at least in part on observing changes in sample deformation.

The sample holders of the invention also, in some embodiments, do not include circuitry for heating and temperature measurement. It will be appreciated that such circuitry in the prior art typically creates paths of high thermal conductivity between the sample and the substrate that can create large temperature gradients across the sample. Therefore, eliminating such circuitry promotes greater temperature uniformity across the sample, improves the thermal isolation of the sample, and makes the sample holder easier to fabricate.

Further improvements in the thermal isolation of the sample are achieved, in some embodiments, by limiting the thermal conductivity of the suspension, the portion of the membrane between the sample region and the substrate. Accordingly, the suspension can be reduced to two segments disposed on opposite sides of the sample region so that the membrane forms a bridge suspended at both ends. Three or more segments can also be used to suspend the sample region. In all of these embodiments limiting the contact area between the suspension and the substrate limits the capacity of the suspension to conduct heat between the sample and the substrate. Still further thermal isolation is achieved in some embodiments by introducing perforations through the suspension. The porosity introduced by such perforations effectively reduces the overall density of the suspension and therefore lowers the thermal conductivity of the suspension. It will be appreciated that sample holders of the invention can include any combination of the foregoing features.

The invention also includes analytical measurement systems that, in some embodiments, incorporate sample holders of the invention, though it will be appreciated that some of the analytical measurement systems of the invention are not limited to the sample holders described herein. Some analytical measurement systems of the invention heat the sample by laser heating, and cool the sample by removing the laser heating. In some of these embodiments the laser is directed at a backing layer of the sample holder, though the backing layer is not essential and in some embodiments the laser is directed at the side of the sample region opposite the side on which the sample is disposed. Other heating methods can also be used, including resistive heating of the sample. The heating methods used by the analytical measurement systems of the invention can address sample holders in an array either serially or in parallel. Parallel heating of multiple array members can be accomplished, for example, through the use of multiple lasers.

The analytical measurement systems of the invention can detect changes in a sample as the sample is heated, cooled, or otherwise acted upon by exposure to reactant chemicals, changing pressures, and so forth. Although phase changes are used throughout this disclosure as examples of changes that can be detected, it will be appreciated that other changes can also be detected. For example, the sample can be a laminate of two different materials and the detected change can be a delamination between the materials. Mechanical changes such as swelling due to moisture absorption, oxidation, or other chemical reactions can likewise be detected. Additionally, the analytical measurement systems of the invention can also detect the extent of completion a chemical reaction. In short, any change in the sample that changes a measurable property can be investigated. Measurable properties can include, but are not limited to, appearance, dimensions, mass, stress, vibrational responses including frequency, amplitude and phase, reflectivity, magnetization, fluorescent yield, electrical resistance, and so forth. Changes in the measurable properties can be correlated to changes in the sample's composition, viscosity, elasticity, crystallinity, phase, surface roughness, refractive index, adhesion to another material, surface tension, heat capacity, etc.

Some analytical measurement systems of the invention are configured to detect changes in the surface of the sample. A property of the surface, such as the topography of the surface, can change abruptly due to a phase change of the sample. For example, the surface may become more or less rough, or may transform from smooth to highly cracked or wavy. Monitoring scattered radiation from the sample surface can reveal such topographic changes. In some embodiments the intensity of the scattered radiation received by a detector is monitored for sudden variations. In other embodiments the surface of the sample is imaged. In some of these embodiments image analysis software is used to review a recording made by the detector for changes in the image of the surface of the sample. Blue light is used in some embodiments to illuminate the sample to avoid interference from blackbody radiation at high temperatures. Appropriate filters between the sample and the detector can filter out the blackbody radiation significantly while still passing scattered blue light.

Another property of the surface, reflectivity, can also change abruptly due to a phase change of the sample. Accordingly, some analytical measurement systems of the invention are configured to detect changes in the reflectivity of the surface of the sample. In these embodiments an illumination source, which can be either a source of coherent or incoherent radiation, illuminates the sample at an angle of incidence and a detector situated at an angle of reflectance approximately equal to the angle of incidence monitors the reflected radiation. In some embodiments the radiation directed at the sample is patterned as an array of spots or lines or other patterns, rather than being simply a single point-source. In these embodiments the pattern and/or intensity of the reflected radiation is monitored for sudden changes that can correlate to a topographic change.

The pattern and/or intensity of the reflected radiation can also vary due to changes in the sample other than the topographic changes already noted. For example, a stressed sample can cause the membrane of the sample holder to bend or warp. In an analytical measurement system in which the illumination source and detector are arranged at equal angles but on opposite sides of an axis normal to the substrate, a warped sample will likely reflect much of the incident radiation away from the detector. However, a phase transition that relieves the stress in the sample will also relieve the warp in the sample which will direct more of the reflected radiation to the detector. Thus, even if the surface topography is unchanged by a phase transition, a change in the amount of stress in the sample is detectable by measuring reflectance. In some embodiments more than one laser beam is reflected off of the sample in order to sample more of the surface than just a single point. In some of these embodiments the detector is a line camera detector.

The intensity of the reflected radiation can also be made to vary by causing the sample to vibrate. Sample vibrations modulate the intensity of the reflected radiation received by the detector. One property that can change with a phase transition and that can be probed with vibrations is viscosity. Specifically, when a material is induced to vibrate, the phase of the induced vibration will lag behind the phase of the driving vibration, and the degree of the lag is a function of both the frequency of the driving oscillation and of the viscosity of the material. Thus, a change in the viscosity will cause a change in the phase delay of the induced vibration. Accordingly, some analytical measurement systems of the invention are configured to detect a phase change by inducing a vibration in the sample, monitoring the modulation frequency of reflected radiation, and by detecting a change in the phase of modulation.

Other analytical measurement systems of the invention detect a change in a sample by monitoring the electrical resistance of the sample, as different phases, for instance, typically have different resitivities. These analytical measurement systems can have sample holders that include electrical contacts in order to make the resitivity measurements. Still other analytical measurement systems of the invention detect a change in a sample by monitoring radiation that is emitted by the sample and that is neither reflected or scattered, for example, blackbody radiation. Detecting blackbody radiation can be performed without illuminating the sample, and therefore such measurement systems do not need to include an illumination source.

It will be appreciated that the various methods described herein for heating samples and for detecting changes can be used together and that the analytical measurement systems of the invention can be configured to simultaneously use any of the possible combinations thereof. Thus, for example, scattered and reflected radiation can be viewed simultaneously and the results of their measurement can be correlated together.

Turning more specifically to the sample holder of the invention, FIGS. 1 and 2 are, respectively, a cross-sectional view and a bottom plan view of a micro sample holder 100 according to one embodiment of the present invention. The cross-section of FIG. 1 is taken along the line 1-1 in FIG. 2. The sample holder 100 includes a support member 102 including an aperture 104, and a membrane 106 spanning the aperture 104. In an exemplary embodiment the membrane 106 is about 3 mm×3 mm. The membrane 106 can also be about 1 mm×1 mm or smaller. In other embodiments the membrane 106 is ¼″×¼″, 1/16″×5/16″, or ½″×3″ or larger. In FIGS. 1 and 2 the membrane 106 supports a sample 108. As discussed in more detail below with respect to methods for using sample holders of the invention, the sample holder 100 does not have to include a sensor because properties of the sample 108 can be measured by non-intrusive means such as, for example, optical measurements of scattered, reflected, or emitted radiation.

As shown in FIG. 1, the support member 102 is significantly thicker than the membrane 106 to hold the membrane 106 taut during processes such as routine handling, deposition of the sample 108, and testing. The membrane 106, on the other hand, is desirably thin to thermally isolate the sample 108 from the support member 102, as well as for other reasons related to the ability to detect changes in the sample, as discussed below. Thermally isolating the sample 108 from the support member 102 advantageously allows the sample 108 to be heated more uniformly due to reduced conduction heat loss to the support member 102. Exemplary materials and dimensions for sample holder 100 will be discussed with respect to the methods of fabrication. It will be appreciated that a single support member 102 can be fabricated with a plurality of apertures 104, each with a membrane 106, to facilitate the testing of sets or libraries of samples. It will be further appreciated that the membrane 106, in some embodiments, can be a cantilever fixed at only one end to the support member 102 within the aperture 104. These embodiments trade some loss in mechanical stability for a further decrease in conduction heat loss to the support member 102.

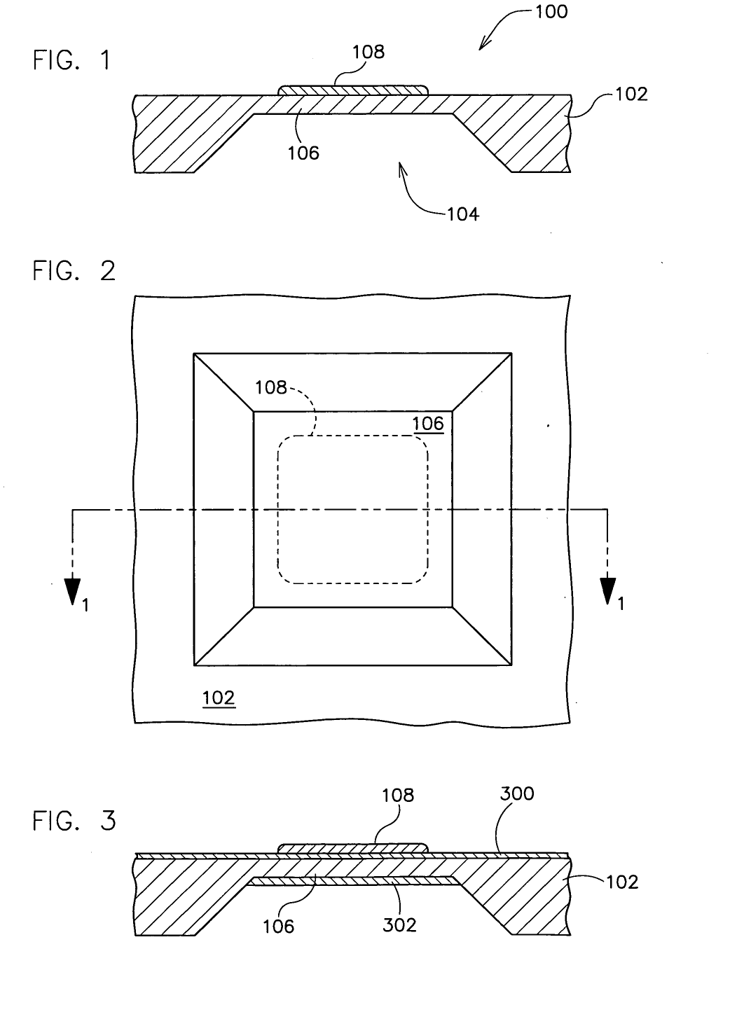

In some embodiments, as shown in the cross-sectional view of FIG. 3, a protective film 300 can be deposited over the membrane 106 to prevent chemical reactions between the sample 108 and the membrane 106. Appropriate materials and thicknesses for the protective film 300 depend on the sample 108, the material selected for the membrane 106, and the deposition and testing conditions for the sample 108. A similar capping layer (not shown) can be formed over the sample to prevent the sample from volatilizing at high temperature and contaminating surrounding samples. The capping layer can also serve to protect the sample from reactive species in the environment (e.g., water, oxygen, etc.) after the sample has been prepared and either before the sample has been tested or during testing. The capping layer may also serve to retain a melted sample.

As also shown in FIG. 3, some embodiments can include an optical coating 302 on a side opposite the side for supporting the sample 108 (hereinafter the “backside”). The optical coating 302, in some embodiments, is of a material that strongly absorbs light of a desired wavelength or range of wavelengths. More particularly, where the membrane 106 is to be used to transfer heat to the sample 108 during a thermal analysis, and a laser is used to heat the membrane 106 from the backside, as will be discussed in more detail below, the optical coating 302 is preferably a material that strongly absorbs the particular frequency of light emitted by that laser. Further, the optical coating 302 preferably has a thickness that is sufficient to absorb a significant amount of the incident light so that an insignificant amount of the light is transmitted through to the membrane 106.

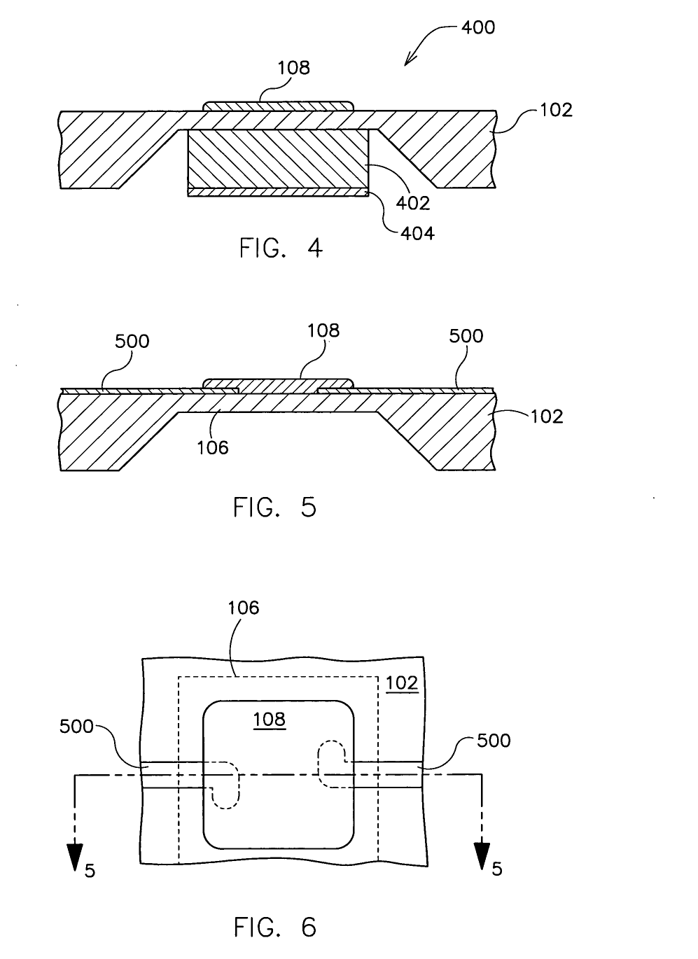

FIG. 4 is a cross-sectional view of another micro sample holder 400 according to another embodiment of the invention. In these embodiments, in addition to the support member 102 and membrane 106, the sample holder 400 also includes a backing layer 402 attached to a backside of the membrane 106. The backing layer 402 is preferably centrally located on the backside. The portion of the membrane 106 that is contiguous with the backing layer 402 forms a sample region of the membrane 106. Portions of the membrane 106 between the support member 102 and the sample region form a suspension that suspends the sample region within the aperture 104 (FIG. 1). The suspension is preferably very thin to provide thermal insulation between the sample 108 and the support member 102. Other methods to further limit the thermal conductivity of the suspension will be discussed below.

The backing layer 402 provides several advantages to the sample holder 400. For example, the backing layer 402 reinforces the sample region so that the sample region is better able to resist distorting or breaking in response to lateral stresses that can be caused by the sample 108. Such stresses can arise as the sample 108 is formed and can also arise during thermal testing due to differences in the coefficients of thermal expansion between the sample 108 and the membrane 106.

Another advantage of the backing layer 402 is that it can uniformly distribute heat to the sample 108. As described above, a laser can be directed onto the backside of the membrane 106 to heat the membrane 106, and in those embodiments in which the sample holder 400 includes the backing layer 402 the laser beam is instead directed onto the backing layer 402. The backing layer 402, heated by the incident laser beam, can then uniformly heat the membrane 106 beneath the sample 108. In these embodiments the backing layer 402 is preferably formed from a material with a high coefficient of thermally conductivity, such as silicon. For essentially the same reasons provided above, the backing layer 402 can also include an optical coating 404 to enhance absorption of the incident laser light.

Embodiments both with and without the backing layer 402 can also include two or more electrical contacts 500 as shown in FIG. 5. The electrical contacts 500 can be formed such that they extend from a top surface of the support member 102 and onto the membrane 106 to terminate within the sample region. In some embodiments four electrical contacts 500 are used to enable a four-point electrical probe, described below. As shown in FIGS. 5 and 6, which respectively show cross-sectional and top views, the sample 108 can be formed above the membrane 106 and the electrical contacts 500 such that the sample 108 is in electrical communication with each of the electrical contacts 500. The use of electrical contacts 500 allows electronic properties of the sample 108 to be measured during an analysis. In those embodiments that include a protective layer 300 (FIG. 3), the protective layer is disposed between the membrane 106 and the electrical contacts 500 so that the electrical contacts 500 can make contact with the sample 108.

Preferably, the size of the electrical contacts 500 is small relative to the size of the sample 108 so that the electrical contacts 500 do not significantly affect the thermal properties of the sample 108 by providing thermally conductive conduits for dissipating heat away from the sample region. The material, shape, and the thickness of the electrical contacts 500 can also be optimized for specific measurements. For example, each contact 500 can be fabricated as a “T” or an “L” to permit a larger contact area between the electrical contact 500 and the sample 108 while keeping the thermally conductive path between the sample 108 and the support member 102 narrow to limit the flow of heat.

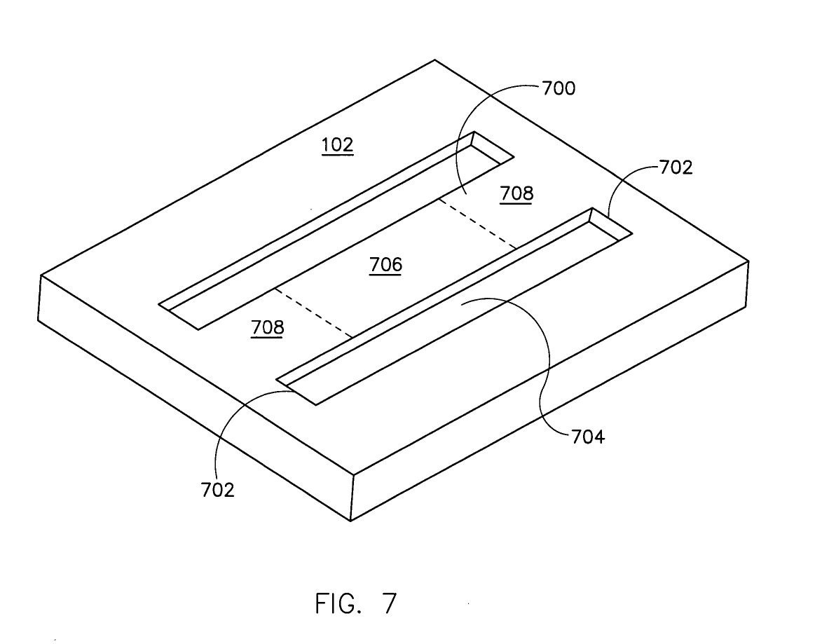

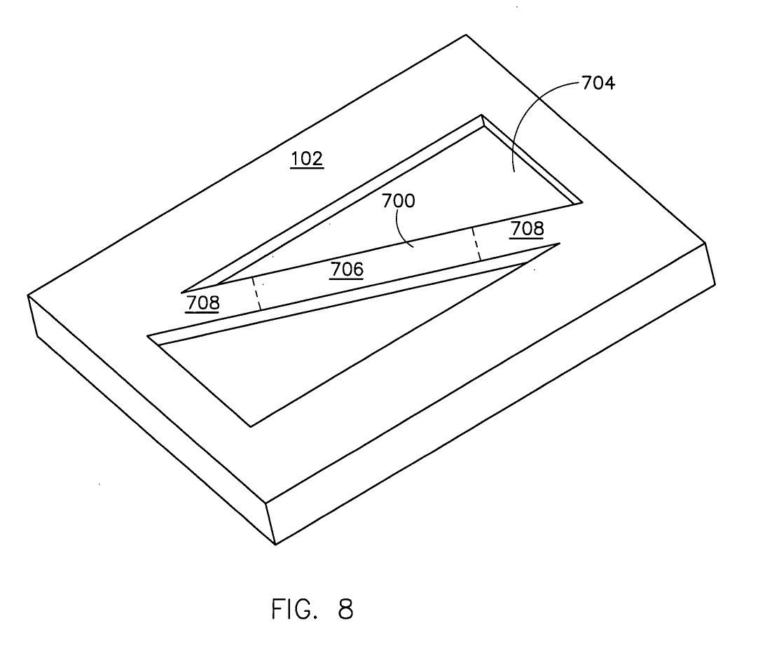

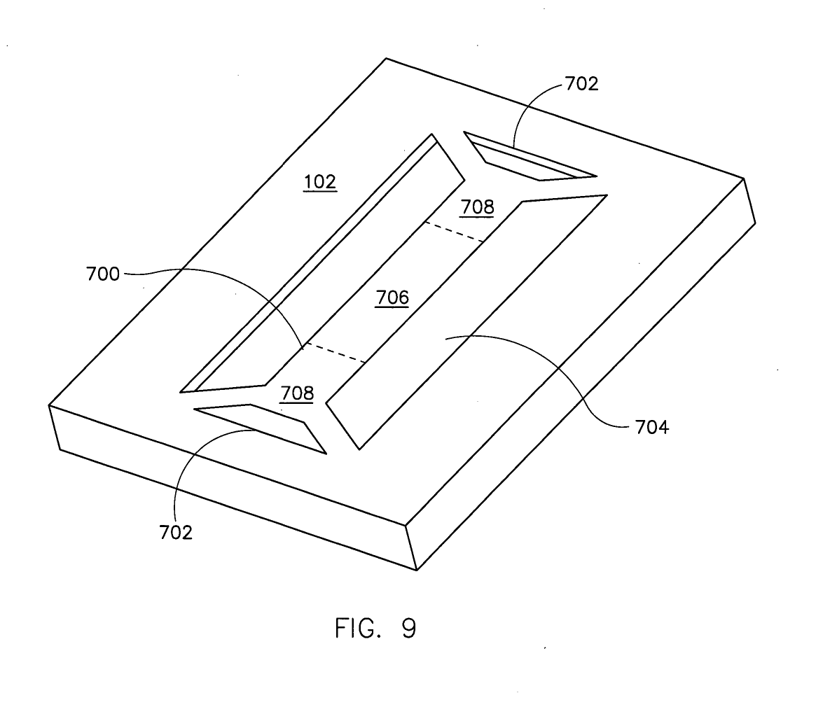

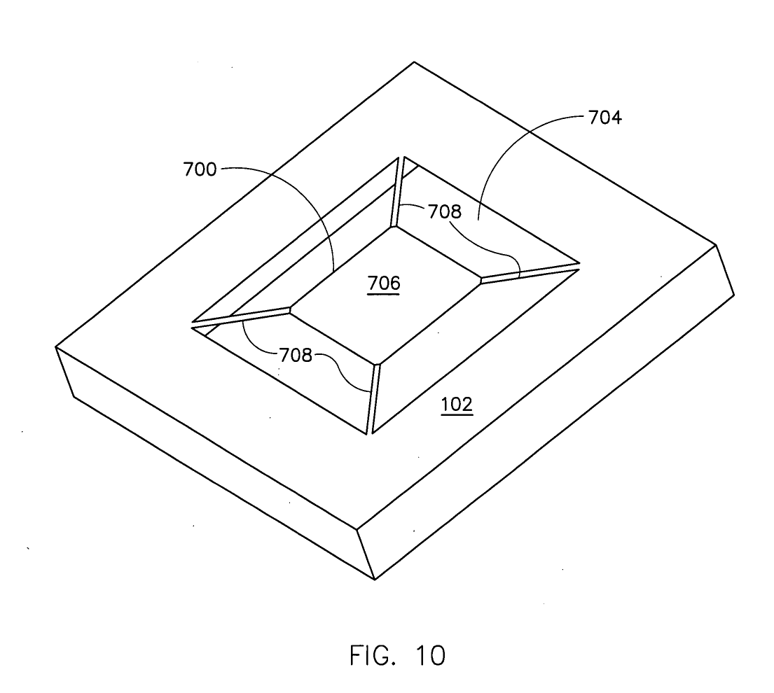

In the above-described embodiments the suspension spans the entire space between the sample region and the support member 102. In other embodiments the suspension spans only a portion of this space. In these embodiments the membrane includes one or more segments between the sample region and the support member 102. FIG. 7 shows a perspective view of an exemplary embodiment in which a membrane 700 forms a bridge between opposing sides 702 of an aperture 704 (or opposite comers of the aperture 704 as in FIG. 8). Advantageously, limiting the suspension in this way reduces the heat conduction across the suspension to the support member 102 to further thermally isolate the sample 108. Additionally, the sample region of the membrane is less mechanically constrained in these embodiments. Therefore, these embodiments provide the sample region with a greater ability to deform in response to sample stresses, and the ability to deform is advantageous in those analytical measurement systems that use reflected radiation to monitor changes in sample deformation as an indication of a change. Likewise, having a less constrained sample region is advantageous in those analytical measurement systems that use reflected radiation to monitor changes in vibration frequency as an indication of a phase change.

In the example shown in FIG. 7, the membrane 700 includes a sample region 706 that is joined to the support member 102 by two segments 708. Although in the present example the segments 708 and the sample region 706 are integral with one another and have the same widths and thicknesses, in other embodiments the segments 708 and sample region 706 are distinguishable from one another, as shown in the alternative exemplary embodiments of FIGS. 8-10. As above, these embodiments can be fabricated to include any combination of an optical coating 302 (FIG. 3), a backing layer 402 (FIG. 4) attached to the backside of the sample region 706, a protective layer 300 (FIG. 3), a capping layer, and electrodes 500 (FIG. 5).

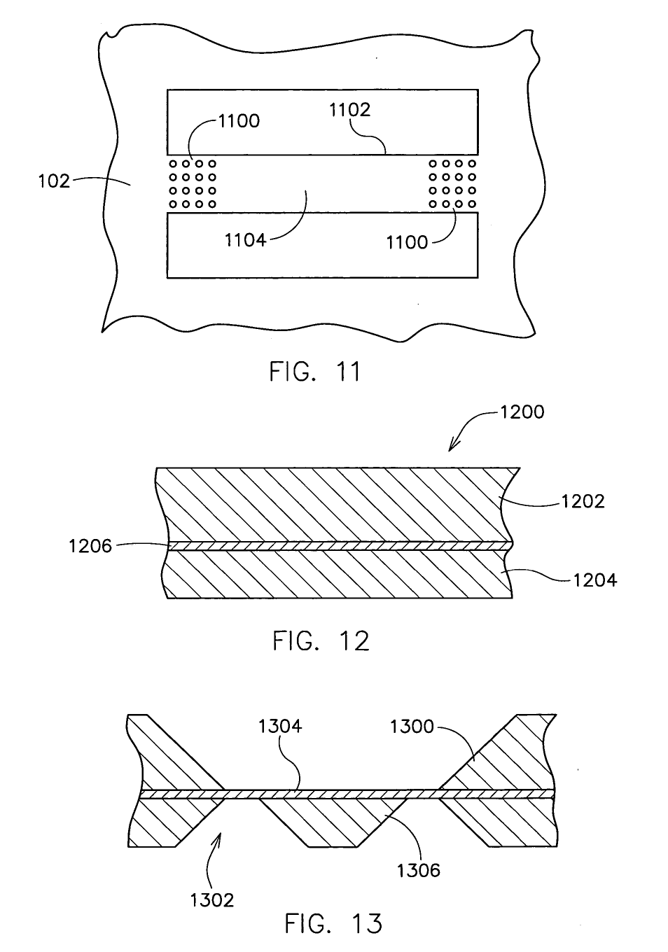

FIG. 11 shows a top plan view of an embodiment similar to that shown in FIG. 7. In the embodiment of FIG. 1, segments 1100 of the membrane 1102 are distinguishable from a sample region 1104 because the segments 1100 include a plurality of perforations extending through the thickness of the membrane 1102. The perforations serve to further decrease heat conduction across the segments 1100 from a sample on the sample region 1104 to the support membrane 102. This benefit is particularly important where the segments 1100 have appreciable width. Although segments 1100 can be manufactured with widths substantially less than the width of the sample region 1104 (as in FIG. 10, for example), wider segments 1100 improve the mechanical stability of the membrane 1102 as a whole. However, a wider segment 1100 will conduct more heat than a narrow segment 1100. Thus, one way in which to maintain the mechanical stability of wider segments 1100 without incurring higher heat conduction is to employ perforations in the segments 100. It is also found that perforations in the segments 1100 allow the segments 1100 to better accommodate stresses without kinking or breaking.

Attention will now be directed to exemplary methods of the invention for fabricating the micro sample holders described above. FIG. 12 shows a cross-sectional view of a silicon on insulator (SOI) wafer 1200 that in some embodiments can serve as the starting point for fabricating a micro sample holder. The exemplary SOI wafer 1200 includes a thick single-crystal silicon substrate 1202, a thin silicon overlayer 1204, and a thin SiO₂ layer 1206 between the silicon layers 1202, 1204. Typical thicknesses for the silicon overlayer 1204 range between about 5μ and about 100μ. In some embodiments the silicon overlayer 1204 is desirably very thin and for these embodiments the thickness of the silicon overlayer 1204 is between about 5μ and about 10μ. In other embodiments the thickness of the silicon overlayer 1204 is between about 20μ and about 45μ. Typical thicknesses for the SiO₂ layer 1206 range between about 0.2μ and about 2.0μ. In some embodiments the thickness of the SiO₂ layer 1206 is about 1.0μ. SOI wafers 1200 having layer thicknesses in the ranges given above are commercially available.

Standard photolithographic and etching techniques can be used to pattern the SOI wafer 1200 and to form a support member 1300 having an aperture 1302 and a membrane 1304 spanning the aperture 1302 and formed from the SiO₂ layer 1206. In some embodiments a backing layer 1306 is additionally formed from the silicon overlayer 1204 at or near the center of the membrane 1304. Additional photolithographic or physical masking techniques and film growth steps can be used to form the optical coatings, protective layers, and electrodes described above with reference to the micro sample holders of the invention.

It should be noted that the SiO₂ layer 1206 forms a suitable etch stop for some etchants used to etch through the silicon layers 1202, 1204. Accordingly, fabricating a micro sample holder from a SOI wafer 1200 is desirable because the etching of the silicon layers 1202, 1204 stops at the surfaces of the SiO₂ layer 1206 to leave the membrane 1304 with a uniform thickness and smooth surfaces. It will be appreciated, however, that the device shown in FIG. 13 can also be fabricated from a non-layered substrate, such a single-crystal silicon wafer, by carefully controlling the etching. In these embodiments over etching can completely etch away the membrane 1302, while under etching will create a membrane 1306 that is undesirably too thick.

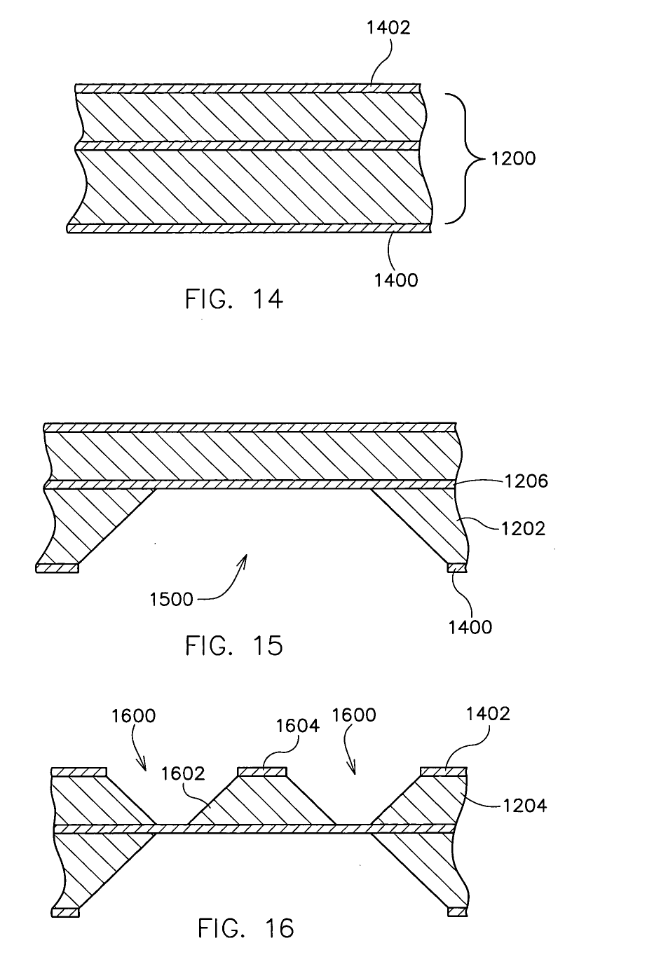

In other embodiments the SOI wafer 1200 is initially provided with first and second nitride layers 1400 and 1402 as shown in FIG. 14. The nitride layers 1400, 1402 can be formed, for example, by subjecting the SOI wafer 1200 to a nitriding process such as a well known chemical vapor deposition (CVD) process that produces a low stress non-stoichiometric silicon nitride film. Next, as shown in FIG. 15, a window 1500 is opened in the first nitride layer 1400 to expose portions of the silicon substrate 1202. Next, the silicon substrate 1202 is etched down to the SiO₂ layer 1206. Thereafter, as shown in FIG. 16, at least two windows 1600 are opened in the second nitride layer 1402 to expose the silicon overlayer 1204, and then the silicon overlayer 1204 is etched down to the SiO₂ layer 1206. A backing layer 1602 is thus formed at this step, as shown.

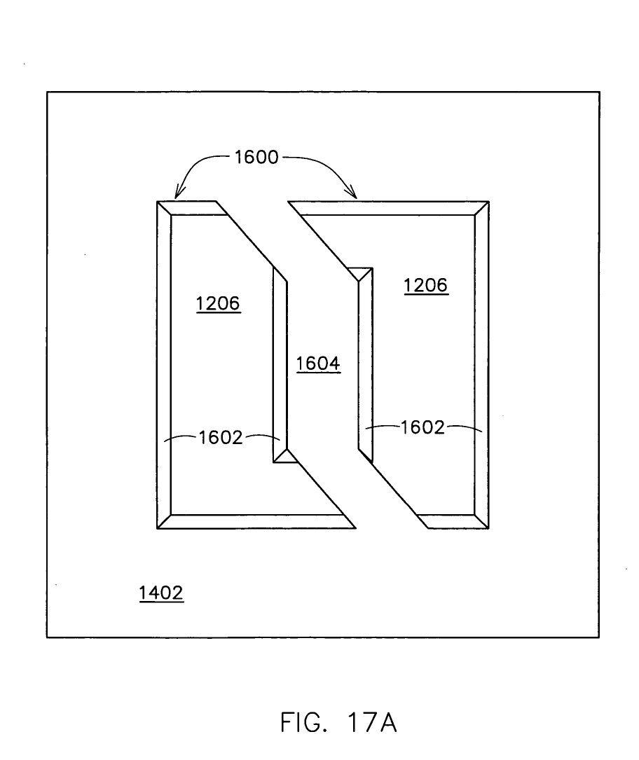

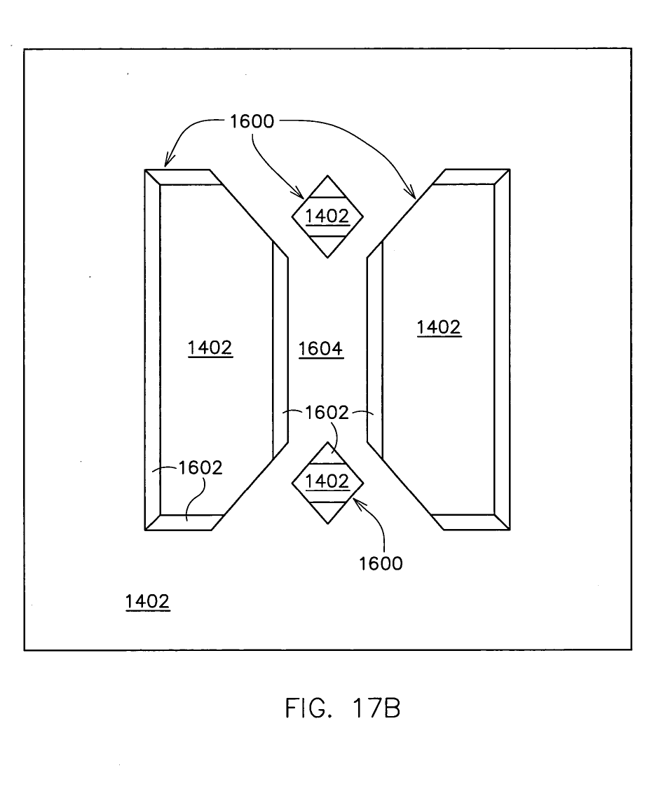

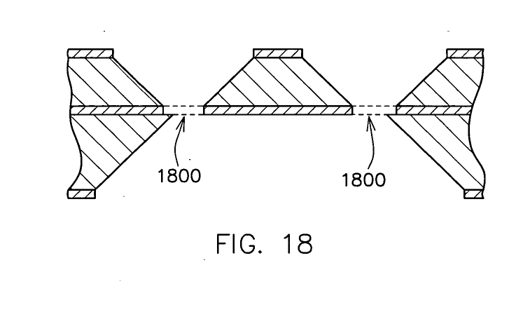

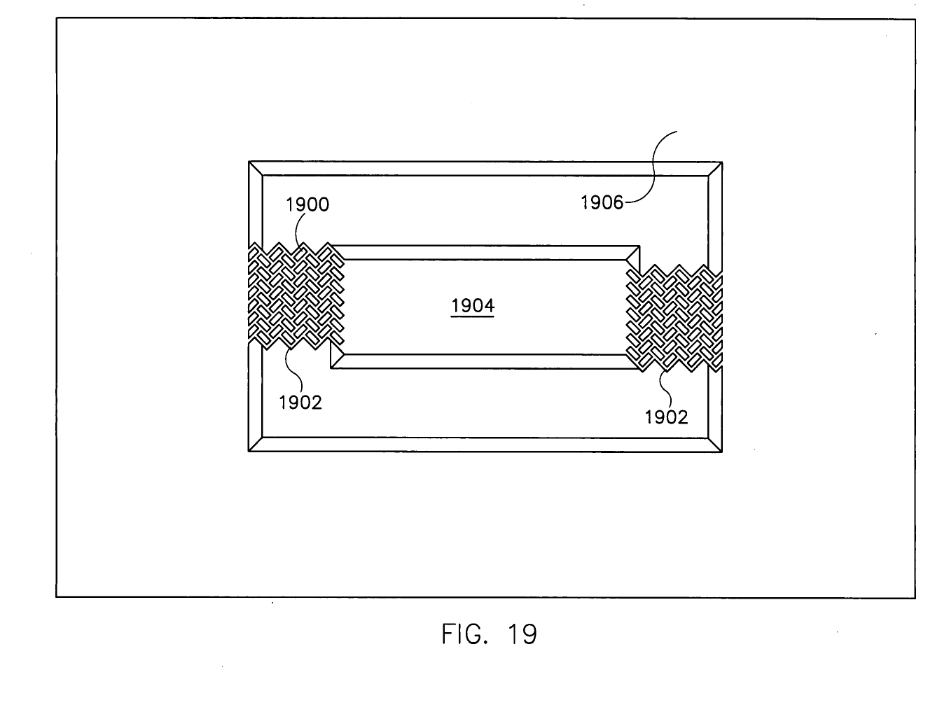

In contrast to the previously described embodiments shown in FIGS. 12 and 13, in the embodiments illustrated by FIGS. 14-16 a membrane 1604 of the micro sample holder is formed from the nitride layer 1402 above the backing layer 1602 rather than from the SiO₂ layer 1206. Therefore, it will be appreciated that in these embodiments the at least two windows 1600 are separated by the portion of the nitride layer 1402 that will form the membrane 1604, as shown in FIGS. 17A and 17B. It can be seen from FIGS. 16, 17A, and 17B that an etch pit is formed in the silicon layer 1204 beneath each of the windows 1600, and each etch pit terminates at the SiO₂ layer 1206. Thus, to complete the micro sample holder the exposed portions of the SiO₂ layer 1206 at the bottom of each etch pit is selectively removed to create the windows 1800 shown in FIG. 18.

FIGS. 17A and 17B show two of many possible arrangements of windows 1600 that can be used to create a suspended membrane 1604. Both of these exemplary embodiments rely on the anisotropic etching of single-crystal silicon to remove the silicon layer 1204 beneath the membrane 1604 to form the suspension between the support member and the sample region.

It will be understood that the advantage of using the SiO₂ layer 1206 as an etch stop, as described above with respect to FIGS. 12 and 13, is equally applicable in these embodiments. However, as above, in other embodiments the SOI wafer 1200 is replaced with a non-layered substrate, such as a single-crystal silicon wafer and carefully controlled etching is employed. Likewise, in some embodiments additional photolithographic steps can be used to form optical coatings, protective layers, and electrodes. It should also be noted that although the embodiments illustrated by FIGS. 14-18 have been described with reference to layers of silicon, SiO₂, and silicon nitride, layers of other materials can be readily substituted for these to match different membrane materials with different backing layer materials, for example.

FIG. 19 is a top plan view of an exemplary embodiment of the micro device of the invention having perforations 1900 in segments 1902 of the membrane between the sample region 1904 and the support member 1906. Perforations 1900 can be formed using standard photolithographic techniques first with an etchant that selectively etches the particular material of the membrane and then with an etchant that selectively etches the particular material of the underlying material. These etching steps can be performed concurrently with other steps described above with reference to FIGS. 12-18. For example, with reference to FIG. 16, perforations 1900 can be formed when the windows 1600 are formed. Thereafter, when the etch pits in the silicon overlayer 1204 that are beneath the windows 1600 are formed, the silicon overlayer 1204 beneath the perforations 1900 can be removed by etching through the perforations 1900.

In some embodiments the perforations 1900 have a rectangular cross-section, or are arranged in a periodic pattern such as the illustrated herringbone pattern, or both. The illustrated pattern in FIG. 19 is particularly well suited to those embodiments in which the membrane is formed above a layer of silicon and the pattern can be aligned with a predominant crystallographic axis of the silicon layer. It will be appreciated that the size of the perforations, the pattern of their arrangement, and the spacing between adjacent perforations can all be varied to produce segments 1902 with differing properties such as strength, spring constant, heat conduction, and so forth, which can be tailored to specific applications.

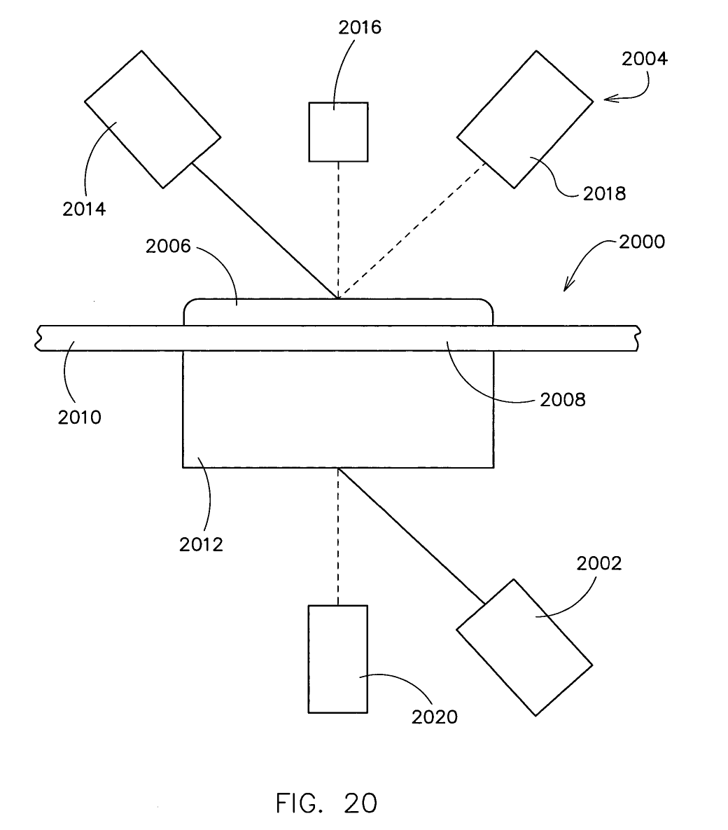

Attention will next be directed to exemplary analytical measurement systems of the invention. FIG. 20 shows a schematic representation of a testing system including a sample holder 2000, a temperature changing device 2002, and a measurement device 2004. In some embodiments the sample holder 2000 is a micro sample holder configured to support a sample 2006 on a sample region 2008 of a membrane 2010, as described above. The temperature changing device 2002 is configured to change the temperature of the sample 2006, for instance, by changing the temperature of the sample region 2008. The measurement device 2004 is configured to measure one or more properties of the sample 2006 as the temperature of the sample 2006 is varied.

In some embodiments the temperature changing device 2002 is a laser that is configured to direct a laser beam towards a backside of the sample region 2008. Directional control of the laser beam can be achieved, for example, with lenses and mirrors to focus and aim the laser beam. In the embodiment illustrated in FIG. 20, the sample holder 2000 includes a backing layer 2012 that receives the laser beam. Although the sample region 2008 can be directly heated by a laser beam in some embodiments, in other embodiments such as the one illustrated, the laser beam heats the backing layer 2012 which then heats the sample region 2008. It should be noted that although the laser beam is illustrated in FIG. 20 as a narrow line directed at the center of the bottom surface of the backing layer 2012, in typical embodiments the laser beam has a width on the order of the width of backing layer 2012. It should also be noted that in some embodiments the temperature changing device 2002 is located above the sample 2006 and directly heats the sample 2006.

Examples of suitable lasers for the temperature changing device 2002 include CO₂ lasers, YAG lasers, and diode lasers. CO₂ lasers produce light with a wavelength of about 10.6μ, a wavelength at which silicon is essentially transparent. Thus, in those embodiments that use a CO₂ laser it is advantageous to use an optical coating 302 (FIG. 3) when the backing layer 2012 is formed of silicon. Suitable optical coatings 302 that strongly absorb light with a wavelength of about 10.6μ include SiO₂, silicon nitride, etc. On the other hand, YAG lasers produce light with a wavelength of about 1.06μ and diode lasers produce light with a wavelength of about 800 nm to 900 nm, and light having these wavelengths is strongly absorbed by silicon or doped silicon. Thus, an optical coating 302 on the backing layer 2012 is not necessary when the temperature changing device 2002 is a YAG or diode laser. Similarly, if the backing layer 2012 is formed of SiO₂ or silicon nitride, and a YAG or diode laser is to be used, an optical coating 302 of silicon is preferred because both SiO₂ and silicon nitride are essentially transparent to the wavelengths produced by these lasers. Other materials that are suitable for the optical coating 302 include carbon black and platinum black.

In some embodiments the laser beam produced by the temperature changing device 2002 is modulated. Modulating the laser beam produces two effects. One effect is to impose a modulation over the rate at which the temperature of the sample 2006 increases. Another effect is to induce a mechanical vibration in the sample 2006. Both effects can improve measurement results as well as provide additional information regarding structural and mechanical properties of the samples.

Although a laser has been used as an example of a temperature changing device 2002, it will be understood that many techniques other than laser irradiation can be similarly employed to change the temperature of the sample 2006. For instance, a photon flux generated by a halogen lamp or infra-red lamp can be directed towards, or focused on, the sample 2006 without filtering. In other embodiments the temperature changing device 2002 is configured to heat the sample 2006 by electrical means. For example, in those embodiments in which the sample holder 2000 includes electrical contacts 500 (FIG. 5), an electric current can be passed through the sample 2006 to heat the sample 2006 by resistive heating. In these embodiments the thermally conductive backing layer 2012 can still serve to distribute heat for more uniform heating of the sample 2006. In some of these embodiments the temperature changing device 2002 is a four-point probe that can be implemented through either two electrical contacts 500 or four electrical contacts 500.

Another technique for electrically heating the sample 2006 is to include a resistive heater on the sample holder 2000. A resistive heater can be a continuous strip of a material with poor electrical conductivity such that it heats rapidly when an electric current is applied. Although not specifically described with reference to the sample holders of the invention, it will be appreciated that a resistive heater can be disposed either between the membrane 2010 and the backing layer 2012, or on the free side of the backing layer 2012. A resistive heater can also be connected between electrical contacts 500. In these latter embodiments an electrically insulating material layer is preferably disposed over the resistive heater to prevent electrical communication with the sample 2006.

As discussed with reference to using a laser for the temperature changing device 2002, electrically heating the sample 2006 can also include a modulation. Modulation can be introduced by modulating the current applied either to the sample 2006 or to the resistive heater, depending on the embodiment. In either case, modulating the applied current can modulate a temperature increase of the sample 2006, and can also cause the sample 2006 to mechanically vibrate.

It will be appreciated that in place of, or in addition to, the temperature changing device 2002, analytical measurement systems of the invention can include devices configured to affect other environmental factors. Accordingly, an analytical measurement system of the invention can also include a vacuum system and a gas delivery system to change the atmosphere surrounding the sample. In these embodiments changes to the sample 2006 can be observed, for instance, at a fixed pressure while the composition of the atmosphere is varied, while the pressure of a reactant gas is varied, or while the pressure of an inert atmosphere is changed. These changes can also be studied as the temperature of the sample is varied or held constant. Likewise, an analytical measurement system of the invention can also include adevice for creating a magnetic field around the sample 2006.

The measurement device 2004 is configured to measure one or more properties of the sample 2006. In some embodiments the measurement device 2004 includes an illumination source 2014 that is configured to illuminate the sample 2006. The illumination source 2014 can be, for example, a laser that produces a laser beam, or a source of non-coherent light such as an LED. In some embodiments in which the illumination source 2014 is a laser, a beam splitting device can be disposed between illumination source 2014 and the sample 2006 in order to generate and/or direct a plurality of laser beams towards the sample 2006. In some of these embodiments the plurality of laser beams are evenly spaced from one another to form an array of laser beams. In some embodiments in which the illumination source 2014 is an LED, the LED can be advantageously configured to emit blue light, as described below. Although lasers and LEDs have been used as examples of the illumination source 2014, in some embodiments the illumination source 2014 generates one of the other common probes used in analytical measurement systems such as X-rays, an electron beam, or an ion beam. For these embodiments the measurement device 2004, described below, must be specially adapted.

The measurement device 2004 also includes one or more devices for receiving an illumination emanating from the sample 2006. One such device can be a camera 2016 aimed towards the sample 2006. The camera 2016 can be, for example, a video camera, a CCD device, or a CMOS device, used to monitor changes in light scattered from the surface of the sample 2006 due to changes in the surface such as a change in roughness. For instance, the surface of the sample 2006 can change as the sample 2006 undergoes a phase transition at a phase transition temperature such as the glass transition temperature, Tg, the crystallization temperature, Tx, or the melting point, Tm. A recording of the images of the sample 2006 made as the sample 2006 is heated and/or cooled can be correlated to the temperature of the sample 2006 and then either reviewed visually for changes that indicate the occurrence of a phase change or reviewed electronically, for instance, by software configured to compare successive frames in a video recording for changes.

As noted, there are advantages to using blue light to illuminate the surface of the sample 2006. This can best be understood by first noting that the sample 2006 produces blackbody radiation over a range of wavelengths and that range is a function of the temperature of the sample 2006. As the sample 2006 is heated, the center of the blackbody radiation range moves to shorter wavelengths from the infra-red portion of the spectrum increasingly into the red portion of the spectrum. Thus, a very hot sample 2006 can be seen to glow red, and the intensity of the glow as the sample 2006 is increasingly heated can outshine the scattered light from the illumination source 2014. However, an appropriate filter (not shown) disposed between the sample 2006 and the camera 2016 substantially blocks most of the visible portion of the blackbody radiation detected by the camera 2016 without blocking shorter wavelengths. Accordingly, blue light works well to illuminate the sample 2006 because scattered blue light passes through the filter to reach the camera 2016. Some filters allow only a specific window of wavelengths to pass, and a suitable filter for passing blue light would have a window centered at the corresponding wavelength, e. g., about 475 nm. It will be appreciated that other colors of light can also be used, however, the greatest separation between the wavelength of the illumination and the wavelength of blackbody radiation is most preferred. Thus, for example, violet light is more desirable than blue, which is more desirable than yellow.

Another device that can be included in the measurement device 2004 for receiving illumination from the sample 2006 is a light detector 2018 aimed towards the sample 2006. A simple light detector can be used to monitor the intensity of the light reflected from the surface of the sample 1806 in the direction of the light detector 2018. The intensity of the reflected light can vary for many reasons. For example, mechanical vibrations of the sample 2006 will modulate the intensity of the reflected light received by the light detector 2018. Additionally, bending or warping of the sample 2006 will cause the intensity of the reflected light received by the light detector 2018 to change. Likewise, changes in the reflectivity of the surface of the sample 2006 will cause a change in the intensity of the reflected light received by the light detector 2018. As discussed below, more complex light detectors 2018 are able to monitor more than simply an integrated overall intensity and can instead monitor spatial intensity variations so as to be able to monitor variations in light patterns. Monitoring the change in a pattern of light reflected from the sample 2006 can be a more reliable and informative measure of a change in the sample.

Each of the above-described changes in reflectivity can be correlated to phase transitions. For instance, an imposed mechanical vibration will cause a material to vibrate in response thereto. The responsive vibration will have a component at the same frequency as the imposed vibration and may additionally have components at higher harmonics. The phases of the components of the responsive vibration is a function of the frequency of the imposed vibration and also a function of the viscoelastic properties. Accordingly, when a vibrating amorphous material is heated beyond Tg, and transforms from an elastic low temperature state to a viscoelastic high temperature state, a shift in the phase of the responsive vibration is observed. Similar vibration transitions are observable for other phase transitions.

It should be noted that mechanical vibrations can be induced in the sample 2006 through techniques other than modulated heating from either a modulated laser beam or a modulated electrical heating system. For example, a mechanical oscillator (not shown) affixed to the support member 102 (FIG. 1) can induce a vibration in the support member 102 that is transmitted to the sample holder 2000 and to the sample 2006. Similarly, vibrations can be induced acoustically by propagation of sound waves through a suitable (e.g. gas) medium. It should also be noted that the magnitude of the frequency change of a responsive vibration due to a phase transition is itself a function of the frequency of the responsive vibration. Accordingly, it is desirable that the source of the imposed vibration allow for tuning of the frequency of the imposed vibration so that the responsive vibration can be adjusted to one that is sensitive to the phase transition of interest. Thus, in some embodiments, as the temperature of the sample 2006 is varied the sample 2006 is also repeatedly subjected to a sweep through a range of vibrational frequencies.

Phase transitions can also increase or decrease the stress in a sample 2006 to cause a deformation such as bending or warping, or a relaxation of a preexisting deformation. Such bending or relaxation of the sample 2006 changes the curvature of the surface of the sample 2006 and accordingly changes the intensity of the reflected illumination received by the light detector 2018. Therefore, a change in the intensity of a reflected illumination can be correlated to a phase transition. Phase transitions can cause changes in surface reflectivity, such as due to a change in surface roughness. A change in surface reflectivity will also change the intensity of the reflected illumination received by the light detector 2018 in a way that can be correlated back to an occurrence of the phase transition.

In some embodiments a beam splitting device is employed to split the laser light from the illumination source 2014 into a pattern of laser beams, as previously noted. In some embodiments, for example, the pattern is a series of parallel line segments, while in other embodiments the pattern is an array of spaced dots, concentric circles, or other conceivable patterns of light. In some of these embodiments the light detector 2018 is a line camera detector in which the imaging array is a single row of pixel sensors. In some other embodiments, the light detector 2018 is an area camera detector in which the imaging array is a rectangular array of pixel sensors. These embodiments are desirable for monitoring changes because the pattern of laser beams is effective to monitor multiple points on the surface of the sample 2006 and these camera detectors are effective to monitor multiple reflected beams. Other detectors and arrangements are also possible, for example an array of multiple single detectors, which in some cases may provide higher sensitivity, faster response, lower cost, or other special features.

More particularly, in some embodiments, the illumination source 2014 is split into a number of beams that, when projected onto a flat surface, create a series of parallel line segments. The parallel line segments define a plane that is perpendicular to themselves. In these embodiments the line camera detector is oriented such that the row of pixel sensors is aligned with the plane perpendicular to the line segments. When the series of parallel line segments are projected onto the sample 2006, the line segments define an axis on the sample 2006 that is also aligned with the plane perpendicular to the line segments. It will be appreciated that an image of the line segments as seen by the line camera (in other words, the locations on the row of pixel sensors where the reflected line segments intersect the row) will be insensitive to rotation of the sample 2006 about the axis. On the other hand, a change in the curvature of the sample 2006 along that axis will change where the reflected line segments cross the row of pixel sensors. Thus, the image of the line segments, which is merely a series of dots, will show the dots moving closer together or further apart as the curvature of the sample 2006 varies. If the sample 2006 is induced to vibrate, the spacings between the dots in the image of the line segments will oscillate with the frequency of the vibration and any harmonics. A further advantage of the line camera is the high frame rate that is achievable, which allows the high frequency changes to be captured.

Other configurations for the measurement device 2004 are also possible. For example, in those embodiments in which the sample holder 2000 includes two or more electrical contacts 500 (FIG. 5) the resistance of the sample 2006 can be measured as the temperature of the sample 2006 is varied. In some embodiments this is achieved with a four-point probe configuration. In another configuration the light detector 2018 is configured to view the backside of the membrane 2010 to monitor illumination from the illumination source 2014 that is transmitted through the sample 2006. In these embodiments the membrane 2010 and the backing layer 2012 are preferably made of materials that are substantially transparent to the wavelength of the illumination from the illumination source 2014.

In still other embodiments the measurement device 2004 is configured to monitor transmitted light in a reflectance mode. More specifically, if the sample 2006 is at least partially transparent to the illumination from the illumination source 2014, some of the illumination will travel through the sample 2006, reflect off of the bottom surface of the sample 2006, and then travel back through the sample 2006 before reaching the light detector 2018. In these embodiments the measurement device preferably includes a Fourier-Transform Infra-Red spectrometer. Also, in these embodiments a highly reflective material layer is disposed beneath the sample 2006 to increase the amount of the illumination that it reflected back through the sample 2006.

In some embodiments the measurement device 2004 also includes a temperature detector 2020 to measure the temperature of the sample 2006 so that the temperature of the sample 2006 can be controlled in a closed-loop fashion in real time. In embodiments that do not include a temperature detector 2020, property measurements can still be correlated against temperature via a pre-calibrated heating-cooling profile versus time.

In those embodiments that do include temperature detector 2020, the temperature detector 2020 can be, for example, an infra-red detector. The infra-red detector is preferably configured to receive blackbody radiation emitted from the backing layer 2012 to measure the temperature of the backing layer 2012 rather than the temperature of the sample 2006 directly. This is desirable because the emissivity of the backing layer 2012 material as a function of temperature is likely to be known, whereas the same is generally not true for the sample 2006. It should be noted that in some embodiments emissivity from the sample 2006 is measured as a function of changing temperature, and in these embodiments the camera 2016 can be an infrared camera containing an infrared focal plane array or a regular IR sensor. Also, in these embodiments it is sufficient to heat and cool the sample 2006 to change the emissivity therefrom, and consequently illumination source 2014 is not a necessary component of the measurement device 2004.

It will be appreciated that in some embodiments more than one of the above measurement techniques are employed. For example, in some embodiments a camera 2016 monitors scattered light, a light detector 2018 monitors reflected light, electrical resistance of the sample 2006 is measured between electrical contacts 500 (FIG. 5), and the temperature is monitored with a temperature detector 2020. It will also be appreciated that although the micro sample holder of the invention provides many advantages, analytical testing systems of the invention are not limited to those that include the micro sample holder. For example, in some embodiments the sample holder 2000 is a polished substrate of an appropriate material (e.g. fused SiO₂) having appropriate thickness. In these embodiments a laser is used to heat the backside of the substrate while properties such as electrical resistance and vibrational response of the sample 2006 are monitored. It will be further appreciated that a plurality of samples 2006 can be supported on a single substrate and then either tested sequentially, in sets of several samples, or all at the same time. In those embodiments in which more than one sample 2006 is tested at the same time, additional temperature changing devices 2002 and measurement devices 2004 are employed in parallel.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated herein by reference for all purposes.

1. An analytical measurement system comprising:
a micro sample holder including
a support member having an aperture disposed therein,
a membrane spanning the aperture and having a sample region and including
a first surface for supporting a sample on the sample region, and
a second surface opposite the first surface, and
a thermally conductive backing layer in thermal communication with the second surface and contiguous with the sample region of the membrane;
a temperature changing device configured to change a temperature of the sample over a measurement period of time; and
a measurement device configured to measure a change in a property of the sample over the measurement period.

2. The analytical measurement system of claim 1 wherein the sample region of the membrane is centrally located.

3. The analytical measurement system of claim 1 wherein the backing layer includes a coating for absorbing light.

4. The analytical measurement system of claim 1 further comprising at least two electrical contacts disposed on the first surface for electrical communication with the sample.

5. The analytical measurement system of claim 4 wherein the temperature changing device is configured to pass a current between the at least two electrical contacts so as to heat the sample.

6. The analytical measurement system of claim 5 wherein the temperature changing device is further configured to modulate the current.

7. The analytical measurement system of claim 4 wherein the measurement device is in electrical communication with the at least two electrical contacts and the change in the property of the sample measured by the measurement device includes a change in electrical resistance.

8. The analytical measurement system of claim 1 further comprising a protective film disposed over the membrane to prevent chemical reactions between the sample and the membrane.

9. The analytical measurement system of claim 1 wherein the membrane forms a bridge between opposing sides of the aperture.

10. The analytical measurement system of claim 1 wherein the membrane further includes a suspension having at least two segments disposed between the sample region and the support member.

11. The analytical measurement system of claim 1 wherein the at least two segments include a plurality of perforations.

12. The analytical measurement system of claim 1 wherein the measurement device measures the change in the property of the sample by recording the property as a function of time as the temperature of the sample is changed.

13. The analytical measurement system of claim 1 wherein the measurement device measures the change in the property of the sample by recording the property as a function of temperature as the temperature of the sample is changed.

14. The analytical measurement system of claim 1 wherein the temperature changing device includes a resistive heater.

15. The analytical measurement system of claim 1 wherein the temperature changing device includes a laser configured to direct a laser beam at the backing layer.

16. The analytical measurement system of claim 15 wherein the laser is configured to modulate the laser beam.

17. The analytical measurement system of claim 1 wherein the measurement device is configured to record illumination scattered from the sample.

18. The analytical measurement system of claim 1 wherein the measurement device is configured to record illumination reflected from the sample.

19. The analytical measurement system of claim 1 wherein the measurement device is configured to record radiation emitted by the backing layer.

20. The analytical measurement system of claim 1 wherein the measurement device further includes a mechanical oscillator affixed to the support member to harmonically induce a vibration in the sample.

21. The analytical measurement system of claim 20 wherein the mechanical oscillator is tunable to vary a frequency of the harmonically induced vibration.

22. The analytical measurement system of claim 1 wherein the membrane includes a cantilever.

23. An analytical measurement system comprising:
a sample holder including
a first surface having at least two electrical contacts disposed thereon for electrical communication with a sample disposed on the first surface, and
a second surface opposite the first surface;
a laser configured to heat the sample through a temperature range; and
a measurement device configured to measure the electrical resistance of the sample while the sample is heated through the temperature range.

24. The analytical measurement system of claim 23 wherein the laser is configured to heat the sample by directing a laser beam at the second surface of the sample holder.

25. The analytical measurement system of claim 24 wherein the laser is further configured to modulate the laser beam.

26. The analytical measurement system of claim 23 wherein the measurement device includes an infra-red detector configured to receive radiation emitted from the sample holder to measure a temperature thereof.

27. An analytical measurement system comprising:
a sample holder including
a first surface for supporting a sample disposed thereon, and
a second surface opposite the first surface;
a first laser configured to heat the sample through a temperature range; and
a measurement device including
a second laser configured to irradiate the sample, and
a detector configured to receive radiation emanating from the irradiated sample to detect a change in the sample as the sample is heated through the temperature range.

28. The analytical measurement system of claim 27 wherein the first laser is configured to heat the sample by directing a first laser beam at the second surface of the sample holder.

29. The analytical measurement system of claim 27 wherein the radiation emanating from the irradiated sample includes reflected radiation.

30. The analytical measurement system of claim 29 wherein the detector is configured to detect the change in the sample by determining a change in a stress of the sample.

31. The analytical measurement system of claim 29 wherein the detector is configured to detect the change in the sample by determining a change in a viscosity of the sample.

32. The analytical measurement system of claim 27 wherein the second laser is configured to irradiate the sample with an array of laser beams.

33. The analytical measurement system of claim 32 wherein the detector is a line camera detector.

34. An analytical measurement system comprising:
a sample holder including
a first surface for supporting a sample disposed thereon, and
a second surface opposite the first surface;
a laser configured to heat the sample through a temperature range; and
a measurement device including
a light source configured to illuminate the sample, and
a detector configured to receive radiation emanating from the illuminated sample to detect a change in the sample as the sample is heated through the temperature range.

35. The analytical measurement system of claim 34 wherein the laser is configured to heat the sample by directing a laser beam at the second surface of the sample holder.

36. The analytical measurement system of claim 34 wherein the detector is a video camera.

37. The analytical measurement system of claim 34 wherein the light source produces an illumination with a wavelength shorter than that of infrared radiation.

38. The analytical measurement system of claim 34 wherein the light source is a blue LED.

39. A method for determining a change in a property of a material, the method comprising:
providing a micro sample holder including
a support member having an aperture disposed therein,
a membrane spanning the aperture and having a sample region and also having
a first surface, and
a second surface opposite the first surface, and
a thermally conductive backing layer in thermal communication with the second surface and contiguous with the sample region of the membrane;
synthesizing a sample of the material on the first surface of the sample holder within the sample region;
affecting the environment of the sample while measuring a property of the sample to generate a record thereof; and
analyzing the record for a change in the property.

40. The method of claim 39 wherein affecting the environment of the sample includes changing the temperature of the thermally conductive backing layer.

41. The method of claim 39 wherein affecting the environment of the sample includes changing the pressure of an atmosphere surrounding the sample.

42. The method of claim 39 wherein affecting the environment of the sample includes changing the composition of an atmosphere surrounding the sample.

43. The method of claim 39 wherein measuring the property of the sample includes imaging an appearance of the sample.

44. The method of claim 43 wherein analyzing the record for the change in the property includes correlating a change in the appearance to a phase change.

45. The method of claim 39 wherein measuring the property of the sample includes monitoring a deformation of the sample.

46. The method of claim 45 wherein analyzing the record for the change in the property includes correlating a change in deformation of the sample to a phase change.

47. The method of claim 45 wherein analyzing the record for the change in the property includes correlating a change in deformation of the sample to a change in a composition of the sample.

48. The method of claim 39 wherein measuring the property of the sample includes measuring a vibration of the sample.

49. The method of claim 48 wherein analyzing the record for the change in the property includes correlating a change in the vibration of the sample to a phase change.

50. The method of claim 48 wherein analyzing the record for the change in the property includes correlating a change in the vibration of the sample to a change in the viscosity of the sample.

51. The method of claim 48 wherein analyzing the record for the change in the property includes correlating a change in the vibration of the sample to a change in the mass of the sample.

52. The method of claim 39 wherein measuring the property of the sample includes measuring a reflectivity of the sample.

53. The method of claim 52 wherein analyzing the record for the change in the property includes correlating a change in the reflectivity of the sample to a phase change.

54. The method of claim 39 wherein measuring the property of the sample includes measuring an electrical resistance of the sample.

55. The method of claim 54 wherein analyzing the record for the change in the property includes correlating a change in the electrical resistance of the sample to a phase change.

56. A method for determining a change in a property of a material, the method comprising:
providing a sample holder including a sample region having opposing first and second surfaces;
synthesizing a sample of the material on the first surface of the sample region;
heating the sample region of the sample holder by directing a laser beam towards the second surface while measuring an electrical resistivity of the sample to generate a record thereof; and
analyzing the record for a change in the electrical resistivity.

57. The method of claim 56 wherein analyzing the record for the change in the electrical resistivity includes correlating the change to a phase change.
